(19) United States
(12) Patent Application Publication
(10) Pub. No.: US 2016/0051189 A1
(43) Pub. Date: Feb. 25, 2016

(54) METHOD AND APPARATUS FOR PROCESSING FOOD INFORMATION

(71) Applicant: SAMSUNG ELECTRONICS CO., LTD., Suwon-si (KR)

(72) Inventors: Moonsook LEE, Seoul (KR); Jinyoung PARK, Hwaseong-si (KR); Seongho CHO, Gwacheon-si (KR); Woochang LEE, Anyang-si (KR); Juneyoung LEE, Seongnam-si (KR)

(73) Assignee: SAMSUNG ELECTRONICS CO., LTD., Suwon-si (KR)

(21) Appl. No.: 14/831,472

(22) Filed: Aug. 20, 2015

(30) Foreign Application Priority Data
Aug. 20, 2014 (KR) 10-2014-0108455

(51) Int. Cl. A61B 5/00; G01N 21/35; G01N 22/00; G01N 33/49; G09B 19/00; A61B 5/0402; A61B 5/11; A61B 5/0488; A61B 5/0205; G01N 21/65; A61B 5/145

(52) U.S. Cl. CPC A61B 5/4866; G01N 21/65; G01N 21/35; G01N 22/00; G01N 33/492; A61B 5/7246; A61B 5/14532; A61B 5/14546; A61B 5/4872; A61B 5/0402; A61B 5/1102; A61B 5/0488; A61B 5/0205; A61B 5/443; G09B 19/0092

(57) ABSTRACT

Provided are a method and apparatus for processing food information. The method may include detecting, by a sensor, food information of food consumed by a subject from blood of the subject in a non-invasive manner; and determining, by a processor, a digestive capacity of the subject based on the detected food information.

### CROSS-REFERENCE TO RELATED APPLICATION

This application claims priority from Korean Patent Application No. 10-2014-0108455, filed on Aug. 20, 2014 in the Korean Intellectual Property Office, the disclosure of which is incorporated herein in its entirety by reference.

### BACKGROUND

1. Field

Apparatuses and methods consistent with exemplary embodiments relate to methods and apparatuses for processing food information by detecting food information.

2. Description of the Related Art

Diseases such as diabetes or blood pressure problems are affected by the consumed food. Accordingly, each person may have to have meals appropriate for his/her physical constitution and current health state. A person may use a method of choosing an appropriate diet by directly recording the description and amount of each meal and monitoring a biometric signal so as to figure out the meal appropriate for his/her health state. However, in this case, data may be omitted to be recorded and the entire process of constantly recording such data is quite inconvenient.

Recently, as more people have become interested in health issues and the challenges that adult diseases create, demands for apparatuses for checking health parameters, such as a blood sugar sensor or a blood pressure sensor, have also increased. Thus, there is an increasing need for detecting the food consumed by a person by using these apparatuses.

### SUMMARY

One or more exemplary embodiments provide methods and apparatuses for detecting and processing food information.

Further, one or more exemplary embodiments provide methods and apparatuses for processing bio-signal related to food, as well as food information.

According to an aspect of an exemplary embodiment, there is provided a method of processing food information including: detecting, by a sensor, food information of food consumed by a subject from blood of the subject in a non-invasive manner; and determining, by a processor, a digestive capacity of the subject based on the detected food information.

The food information may include at least one of a type of nutrients and amounts of nutrients in the food consumed by the subject.

The determining the digestive capacity may include calculating a food information pattern that shows a change in the detected food information according to a lapse of time; and calculating the digestive capacity based on the food information pattern. The detecting may be performed by using at least one of a Raman spectroscopy, an infrared spectroscopy, or a radio-frequency (RF) analysis.

The method may further include displaying at least one of the food information and the digestive capacity.

The method may further include detecting bio-signal from the subject; and calculating a correlation between the food information and the bio-signal.

The bio-signal may be detected in a non-invasive manner.

The bio-signal may include at least one of information about blood sugar, cholesterol, or an amount of body fat of the subject, and information about a blood pressure, electrocardiogram (ECG), ballistocardiogram (BCG), photoplethysmograph (PPG), or electromyogram.

The correlation may represent a degree of a change in the bio-signal according to a change in the food information.

The correlation may include a range of the food information which corresponds to a reference range of the bio-signal.

The calculating the correlation may include calculating a food information pattern according to a lapse of time by using the food information; calculating a bio-signal pattern by using the bio-signal; and calculating a value of a correlation between the bio-signal pattern and the food information pattern.

The method may further include detecting at least one of environment information about an external environment of the subject and state information of the subject; and calculating a correlation between the food information, and at least one of the environment information and the state information.

The at least one of the environment information and the state information may include at least one of a temperature, a humidity, a skin moisture content rate of the subject, and a motion of the subject.

According to another aspect of an exemplary embodiment, there is provided an apparatus for processing food information including: a first sensor configured to detect food information of food consumed by a subject from blood of the subject in a non-invasive manner; and a processor configured to calculate a digestive capacity of the subject based on the detected food information.

The food information may include at least one of a type of nutrients and amounts of nutrients in the food consumed by the subject.

The processor may calculate a food information pattern that shows a change in the detected food information according to a lapse of time, and may also calculate the digestive capacity based on the food information pattern.

The first sensor may detect food information by using at least one selected from the group consisting of a Raman spectroscopy, an infrared spectroscopy, or a radio-frequency (RF) analysis.

The apparatus may further include a display module for displaying at least one of the food information and the digestive capacity.

The apparatus may further include a second sensor configured to detect at least one of bio-signal of the subject, environment information about an external environment of the subject, and state information of the subject.

The processor may further calculate a correlation between a result obtained from the second sensor and the food information.

### BRIEF DESCRIPTION OF THE DRAWINGS

The above and/or other aspects will be more apparent by describing certain exemplary embodiments, with reference to the accompanying drawings, in which:

FIG. 1 is a block diagram of an apparatus for processing food information;

FIG. 2 is a flowchart of a method of processing food information according to an exemplary embodiment;

FIG. 3 illustrates an example of a food information pattern according to an exemplary embodiment;

FIGS. 4 through 6 are reference diagrams for explaining a method of providing food information, which is performed by the apparatus for processing food information, according to an exemplary embodiment;

FIG. 7 is a block diagram of an apparatus for processing food information according to another exemplary embodiment;

FIG. 8 is a block diagram of a processor for calculating a correlation between bio-signal and food information according to an exemplary embodiment;

FIG. 9 is a flowchart of a method of calculating a correlation between bio-signal and food information according to an exemplary embodiment; and

FIGS. 10 through 12 are reference diagrams illustrating a correlation between bio-signal and other information according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

As used herein, the term “and/or” includes any and all combinations of one or more of the associated listed items. Expressions such as “at least one of,” when preceding a list of elements, modify the entire list of elements and do not modify the individual elements of the list.

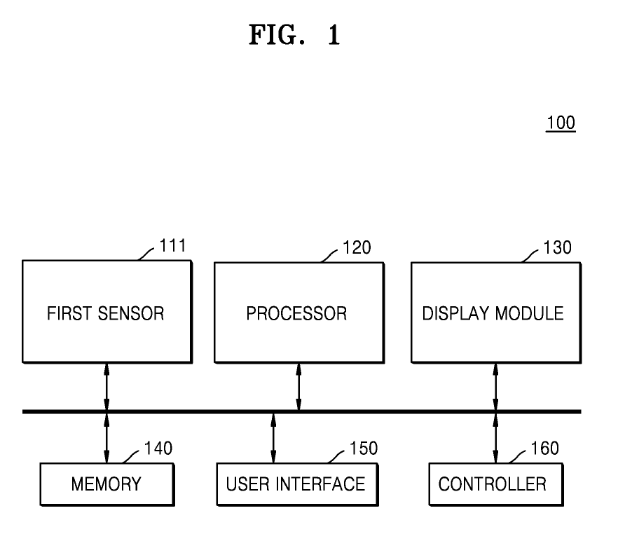

FIG. 1 is a block diagram of an apparatus 100 for processing food information. The apparatus 100 for processing food information may include a first sensor 111 that may detect food information of a subject, a processor 120 that may calculate a digestive capacity of the subject by using the food information received from the first sensor 111, a display module 130 that may display information regarding food, such as food information or a digestive capacity, a user interface 150 that may receive an input of a user command, a memory 140 that may store a program executable by the apparatus 100 for processing food information, and a controller 160 that may control elements in the apparatus 100 for processing food information. The user may be the subject from which food information is to be measured, but the user is a medical expert having an ability to use the apparatus 100. That is, the user may be a broader concept than the subject.

The apparatus 100 for processing food information may be implemented via one housing. The apparatus 100 for processing food information may be a portable apparatus or a wearable apparatus. Alternately, the apparatus 100 for processing food information may be implemented via a plurality of housings. In this case, each element of the apparatus 100 for processing food information may be wired or wirelessly connected to each other. Also, the apparatus 100 for processing food information may be implemented as an element of an apparatus that performs a different function from the apparatus 100 for processing food information, for example, an element of a mobile terminal.

The first sensor 111 may detect information about food consumed by a subject in a non-invasive manner. The first sensor 111 may be implemented with a spectrometer that may optically detect bloodstream and metabolites which are released while and after the subject eats. For example, the first sensor 111 may detect food information by using a Raman spectroscopy, an infrared spectroscopy, or a radio-frequency (RF) analysis. Since substances included in the food may have a different molecular structure from each other according to nutrient components, when light is emitted toward these substances, a wavelength of the light absorbed by the substances may vary with the substances. Thus, food information may be detected by emitting light toward blood of the subject, and analyzing a spectrum of light that is scattered on or reflected from food in the blood. The first sensor 11 may distinguish different nutrients, such as protein, carbohydrates and fat, because each nutrient interacts with light different.

Alternately, when the subject consumes food, a state of the subject is changed. For example, as the subject consumes food, viscosity of blood, a heat flow due to digestion, particularity in blood components, or transparency is changed. Thus, information about the food consumed by the subject may be detected by detecting a state of the subject. If information about food consumed by the subject is predicted by detecting other components instead of directly detecting food components in blood, a database that shows a relation between food and the detected information may be employed.

The first sensor 110 may be worn, for example, on a wrist, a chest, or an ankle of the subject. As the subject consumes food, a bio-signal detected from the subject changes according to the consumed food. An example of the subject may include a person, an animal, or a part of a person or an animal.

The processor 120 may calculate a digestive capacity of the subject by using the detected food information. The processor 120 may calculate a food information pattern by using the detected food information, and calculate a digestive capacity based on the calculated food information pattern. The food information pattern may be a function showing a change in food information according to a lapse of time. The food information pattern may show a change in a total amount of food according to a lapse of time, or a change in each nutrient according to a lapse of time. For example, the processor 120 may receive the detected food information from the first sensor 111. Since a spectrum of scattered or reflected light varies with nutrients included in blood, the processor 120 may calculate an amount of each nutrient distributed in the blood by using a result of a spectrum which is received from the first sensor 111. The processor 120 may calculate a food information pattern that shows a change in each nutrient according to a lapse of time, by calculating an amount of each nutrient according to a lapse of time.

The display module 130 may display information processed by the food information processing apparatus 100. For example, the display module 130 may include a user interface (UI) or a graphical user interface (GUI) for displaying bio-signal. The display module 130 may include at least one of a liquid crystal display (LCD), a thin-film transistor-liquid crystal display (TFT-LCD), an organic light-emitting diode (OLED), a flexible display, and a 3-dimensional (3D) display. According to an implementation type of the apparatus 100 for processing food information, two or more display modules 130 may be present.

The display module 130 and a touch pad for receiving a user input may form a layered structure to constitute a touch screen. If the display module 130 and the touch pad form a layered structure to constitute a touch screen, the display module 130 may be also used as an input unit as well as an output unit. According to an exemplary embodiment, as the display module 130 detects a touch input by a user in a certain area, the display module 130 may automatically start biometric signal measurement.

The memory 140 may store data generated when the apparatus 100 for processing food information performs an operation. According to an exemplary embodiment, the memory 140 is a general storage medium, and it may be understood by those of ordinary skill in the art that the memory 140 may include a hard disk drive (HDD), a read-only memory (ROM), a random-access memory (RAM), flash memory, or a memory card.

The user interface 150 may receive an input for operating the food information processing apparatus 100 from a user, or output at least one of bio-signal, food information, and a correlation which are processed by the food information processing apparatus 100. The user interface 150 may include a button, a key pad, a switch, a dial, or a touch interface so that a user may directly operate the food information processing apparatus 100. The user interface 140 may include a display for displaying an image, and may be implemented as a touchscreen. According to another exemplary embodiment, the user interface 150 may include an input/output (I/O) port that may connect the food information processing apparatus 100 to a human interface device (HID). The user interface 150 may include an I/O port for inputting/outputting an image.

The controller 160 may control all operations of the apparatus 100 for processing food information. For example, the controller 160 may control the first sensor 111 so as to detect food information. Additionally, the controller 160 may determine whether the subject excessively consumed food, whether any nutrient is deficient, or whether food with a low digestion degree is present by analyzing the detected food information and the digestive capacity, and may provide a result via the display module 130.

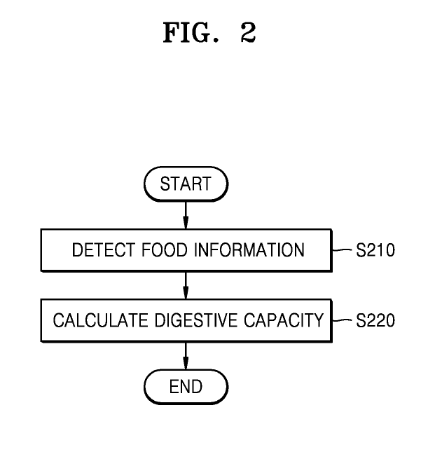

FIG. 2 is a flowchart of a method of processing food information according to an exemplary embodiment. Referring to FIG. 2, in operation S210, the first sensor 111 may detect food information. The first sensor 111 may optically detect information about food consumed by a subject via a non-invasive method. For example, the first sensor 111 may detect food information by using a Raman spectroscopy, an infrared spectroscopy, or an RF analysis given that a wavelength of absorbed light varies with molecular structures of food.

In operation S220, the processor 120 may calculate a digestive capacity of the subject by using the food information received from the first sensor 111. For example, the processor 120 may calculate a food information pattern according to a lapse of time, by using the food information received from the first sensor 111. The food information pattern may include at least one of a pattern showing a change in all nutrients as a whole and a pattern showing a change in each nutrient. Additionally, the processor 120 may calculate a digestive capacity of the subject based on the food information pattern. The processor 120 may calculate a digestive capacity with respect to all nutrients as a whole or a digestive capacity with respect to each nutrient.

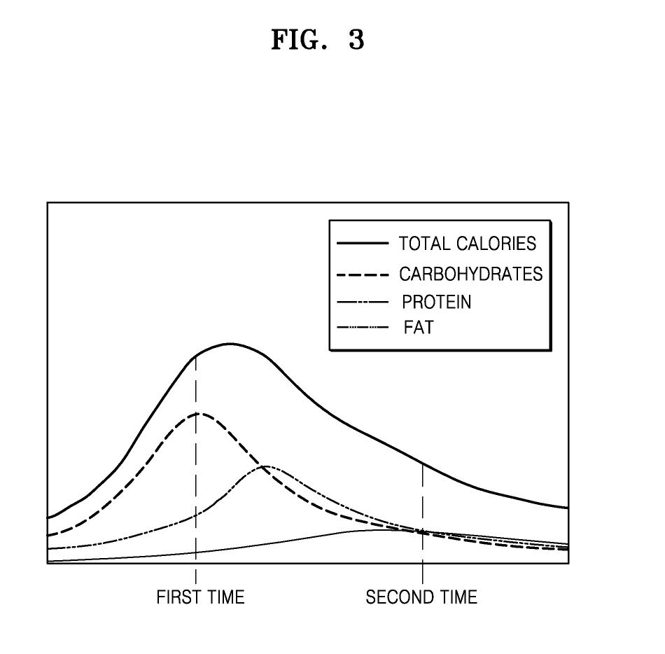

FIG. 3 illustrates an example of a food information pattern according to an exemplary embodiment. The processor 120 may receive a detection result from the sensor 111 and classify food information according to nutrients based on a wavelength bandwidth of absorbed light and calculates an amount of consumed nutrients based on a range of the wavelength bandwidth of the absorbed light. When an amount of each nutrient is calculated, the processor 120 may employ a reference database that represents a relation between the range of a wavelength bandwidth of absorbed light and an amount of each nutrient. Thus, the processor 120 may calculate a food information pattern shown in FIG. 3. As shown in FIG. 3, the food information pattern may include a pattern about total calories, a pattern about carbohydrates, a pattern about protein, and a pattern about fat. A digestive capacity may be defined as an amount of food that remains in the blood at a second time, for example, after a lapse of six hours from a time when food is consumed, compared to an amount of food that remains in the blood at a first time, for example, when the food is consumed. If the digestive capacity is high, the subject digests food well. It may be understood that a subject who has the food information pattern shown in FIG. 3 has a lower digestive capacity of fat, compared to a digestive capacity with regard to carbohydrates.

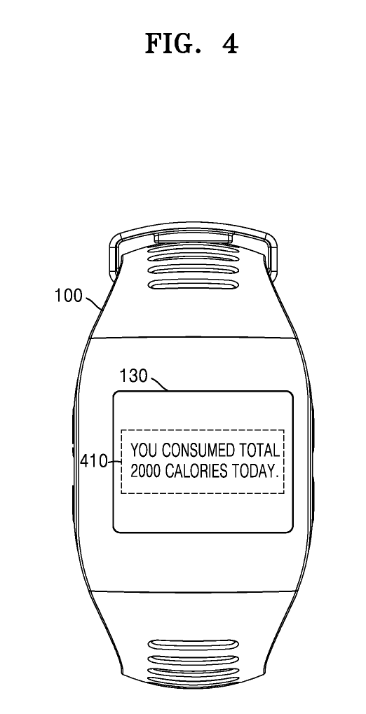

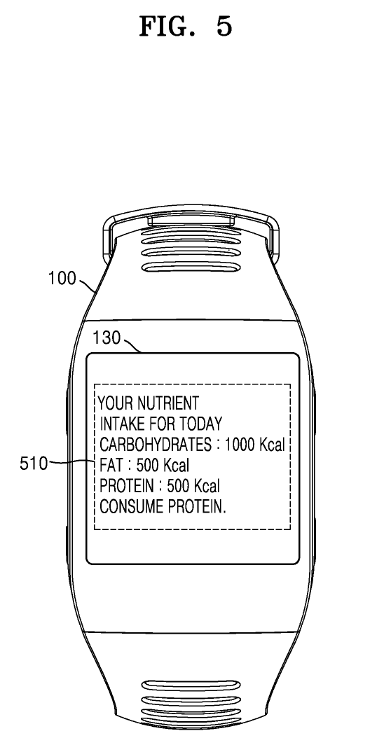

FIGS. 4 through 6 are reference diagrams for explaining a method of providing food information, which is performed by the apparatus 100 for processing food information, according to an exemplary embodiment. As shown in FIG. 4, the apparatus 100 for processing food information may provide an indicator 410 for showing in calories an amount of food consumed by a subject in a certain period of time, for example, in one day. Alternately, as shown in FIG. 5, the apparatus 100 for processing food information may provide an indicator 510 for showing food consumed by the subject with regard to each nutrient. Additionally, the apparatus 100 for processing food information may provide information about a deficient nutrient or an excessively consumed nutrient with reference to a desirable nutrient intake of the subject.

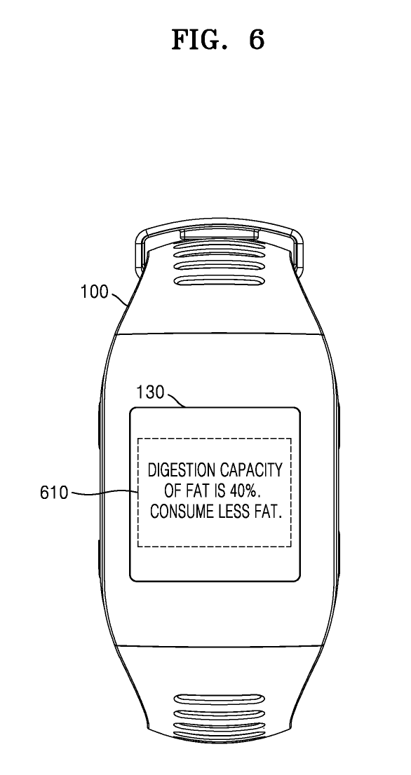

Alternately, as shown in FIG. 6, the apparatus 100 for processing food information may provide the indicator 610 for showing information about a nutrient with a low digestion capacity by calculating a digestion capacity with respect to each nutrient, and thus, may provide a guideline for a food intake of the subject.

The food information may affect bio-signal of the subject. Thus, the food information processing apparatus 100 may calculate a correlation between the food information and the bio-signal of the subject, and provide information about the correlation.

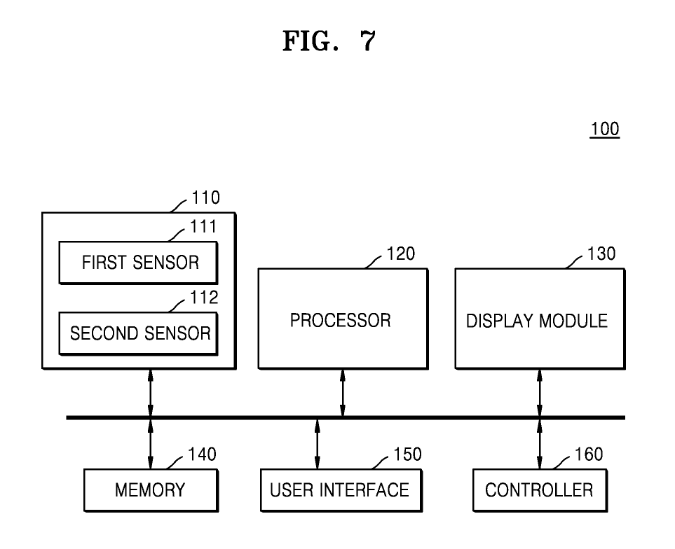

FIG. 7 is a block diagram of the food information processing apparatus 100 according to another exemplary embodiment. Referring to FIGS. 1 and 7, the apparatus 100 for processing food information may further include a second sensor 112 that may detect a bio-signal of the subject. The first sensor 111, the display module 130, the memory 140, and the user interface 150 are described with reference to FIG. 1.

Bio-signal is a unique signal generated from the subject. For example, bio-signal may be a signal that is generated according to a motion of a particular part of the subject such as a heart or muscle, for example, electrocardiogram (ECG), ballistocardiogram (BCG), photoplethysmograph (PPG), electromyogram, a blood pressure, or may be information about substances included in the subject, for example, blood sugar, cholesterol, an amount of body fat.

The second sensor 110 may also detect food information of the subject in a non-invasive manner. The second sensor 112 includes a plurality of electrodes to be in contact with the subject. Thus, the second sensor 112 may detect a bio-signal of the subject by measuring a change in electrical characteristics, for example, a resistance change according to a change in blood. The second sensor 112 may detect a bio-signal by using light instead of an electrode. Since substances included in the subject respectively have a unique molecular structure, a wavelength bandwidth of absorbed light may vary with the substances.

A detection method performed by using the second sensor 112 may vary according to a bio-signal type. For example, if the bio-signal is a signal that is generated according to a motion of a particular part of the subject such as a heart or muscle, for example, ECG, BCG, PPG, electromyogram, or blood pressure, a sensor using electric characteristics may be used as the second sensor 112. If the bio-signal is information about substances contained by the subject, for example, blood sugar, cholesterol, or an amount of body fat, a sensor using light may be employed as the second sensor 112.

The processor 120 may further calculate a correlation between a bio-signal detected by the second sensor 112 and food information stored in the apparatus 100. The bio-signal may be affected by food consumed by the subject. For example, blood sugar may change in correspondence with food consumed by the subject. However, an effect of food on blood sugar may vary with from subject to subject. For example, whereas blood sugar of a subject may be sensitive to food consumed, blood sugar of other subject may be not sensitive to food consumed. Additionally, whereas blood sugar of a subject may be sensitive to carbohydrates included in food consumed, blood sugar of another subject may be sensitive to fat included in food consumed. Accordingly, if information about food that is sensitive to bio-signal for each person is provided, each person may predict a change in his/her bio-signal based on a change in food information.

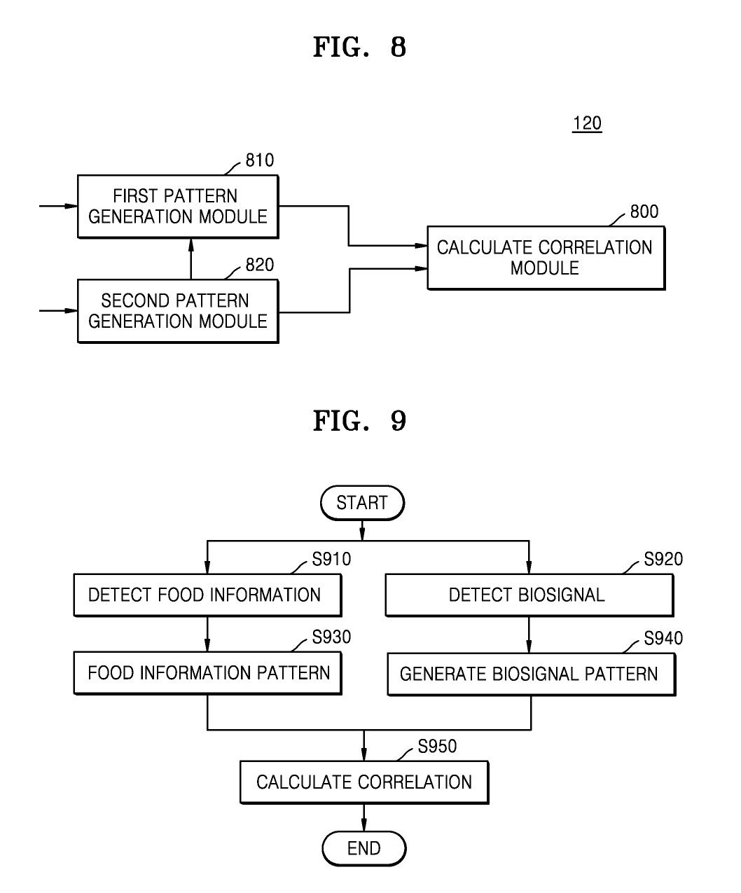

FIG. 8 is a block diagram of the processor 120 for calculating a correlation between bio-signal and food information according to an exemplary embodiment. Referring to FIG. 8, the processor 120 includes a first pattern calculation module 810 that may calculate a food information pattern, a second pattern calculation module 820 that may calculate a bio-signal pattern, and a correlation calculation module 830 that may calculate a correlation between the food information pattern and the bio-signal pattern. Calculation of the food information pattern is identical to a function of the processor 120 which is described with reference to FIG. 1, and thus, a description of the first pattern module 810 is not provided here.

The second pattern calculation module 820 may calculate the bio-signal pattern by using a bio-signal. The bio-signal pattern may be a function showing a change in the bio-signal according to a lapse of time. For example, if the bio-signal is an ECG signal, the second pattern calculation module 820 may amplify an ECG signal received from the second sensor 112 and filter the amplified ECG signal by using a finite impulse response (FIR) bandpass filter. Then, peaks are detected from the filtered ECG signal, and an ECG signal pattern may be calculated by adaptively filtering the detected peaks. Additionally, if the bio-signal carries blood sugar information, the second pattern calculation module 820 may receive the blood sugar information from the second sensor 112 and filter the blood sugar information, and then, calculate a blood sugar information pattern that shows a change in blood sugar according to a lapse of time.

The correlation calculation module 830 may calculate a correlation between the food information and the bio-signal by using the food information pattern and the bio-signal pattern. The correlation may be information about a degree by which food information affects bio-signal. The correlation calculation module 830 may calculate a value of a correlation between the food information pattern and the bio-signal pattern. Additionally, if a correlation value is equal to or higher than a predetermined value, the controller 160 may determine that a correlation between food and bio-signal is high and provide a result of the determining via the display module 130. For example, the correlation calculation module 830 may calculate a correlation between the nutrients as a whole and bio-signal, or calculate a correlation between each nutrient and bio-signal because bio-signal may have a higher correlation with all nutrients as a whole than with each nutrient according to subjects or may have a higher correlation with a particular nutrient than with all nutrients as a whole.

The correlation calculation module 830 may calculate a correlation between a bio-signal pattern and a food information pattern for all time periods. Alternately, the correlation calculation module 830 may calculate a correlation between a bio-signal pattern that is present within a reference range, for example, within an abnormal range, and a food information pattern that corresponds to the bio-signal pattern. For example, the correlation calculation module 830 may calculate food information when a blood pressure is within an abnormal range, and calculate a value of a correlation between the blood pressure within the abnormal range and food information corresponding to the blood pressure, for example, an amount of all nutrients as a whole or an amount of each nutrient. If the correlation value is equal to or higher than a predetermined value, the controller 160 may determine that a correlation between the blood pressure and food is high and display a result of the determining via the display module 130.

The controller 160 may further control the second sensor 112 so as to detect a bio-signal. The controller 160 may display a correlation between food information and the bio-signal on the display module 140, and may also store food information corresponding to the bio-signal within an abnormal range in the memory 140 by using the correlation.

FIG. 9 is a flowchart of a method of calculating a correlation between a bio-signal and food information according to an exemplary embodiment. Referring to FIG. 9, in operation S910, the first sensor 111 may detect food information. The first sensor 111 may optically detect information about food consumed by a subject in a non-invasive method. For example, the first sensor 111 may detect food information by using a Raman spectroscopy, an infrared spectroscopy, or an RF analysis because a wavelength of absorbed light varies with molecular structures of food.

In operation S920, the second sensor 112 may detect a bio-signal by using light or an electrical signal. The bio-signal may include at least one of information about an amount of a substance included in a subject and information about a motion of a part of the subject. For example, bio-signal may include at least one of information about blood sugar, cholesterol, or an amount of body fat and information about a blood pressure, ECG, BCG, PPG, or electromyogram. The second sensor 112 may detect information about an amount of a substance included in the subject by using light, and detect information about a motion of a part of the subject by using a change in an electrical signal.

In operation S930, the first pattern calculation module 810 included in the processor 120 may calculate a food information pattern according to a lapse of time by using the food information received from the first sensor 111. The food information pattern may include at least one of a pattern showing a change in all nutrients as a whole and a pattern showing a change in each nutrient. In operation S940, the second pattern calculation module 820 may also calculate a bio-signal pattern according to a lapse of time by using the bio-signal received from the second sensor 112. When the bio-signal pattern is calculated, detected information may be filtered by using a low-pass filter or an adaptive filter.

In operation S950, the correlation calculation module 830 included in the processor 120 may calculate a correlation between the food information pattern and the bio-signal pattern. The correlation may show a change in the bio-signal pattern according to a change in the food information pattern. The correlation calculation module 830 may calculate a correlation by calculating a value of a correlation between the food information pattern and the bio-signal pattern. The correlation calculation module 830 may calculate a correlation between a bio-signal for each nutrient, or calculate a correlation between all nutrients as a whole and bio-signal. If a correlation value is equal to or higher than a reference value, the controller 160 may determine that a correlation between food information and bio-signal is high. The correlation may include a range of food information that corresponds to bio-signal within an abnormal range. The controller 160 may provide a correlation between the bio-signal for each nutrient via the display module 130.

It has been described above that food information affects bio-signals detected from the subject. However, the embodiments are not limited thereto, and a bio-signal may affect food information. For example, if a digestive capacity of the subject decreases when the subject's blood pressure is high, the apparatus 100 for processing food information may provide information showing that the subject may consume a small amount of food when the subject's blood pressure is high.

Also, subject state information, for example, environment information such as an external environment of a subject or a motion of a subject, may have a correlation with food information. In this case, the apparatus 100 for processing food information may further include a sensor that may detect environment information or subject state information. Environment information may be a temperature, humidity, a skin moisture content rate. A sensor for detecting a motion of the subject may be an acceleration sensor, a gyro sensor, or a terrestrial magnetic sensor. The processor 120 may generate an environment information pattern according to a lapse of time and a state information pattern according to a lapse of time and correlate a result of the generating with food information pattern, so as to calculate a correlation therebetween.

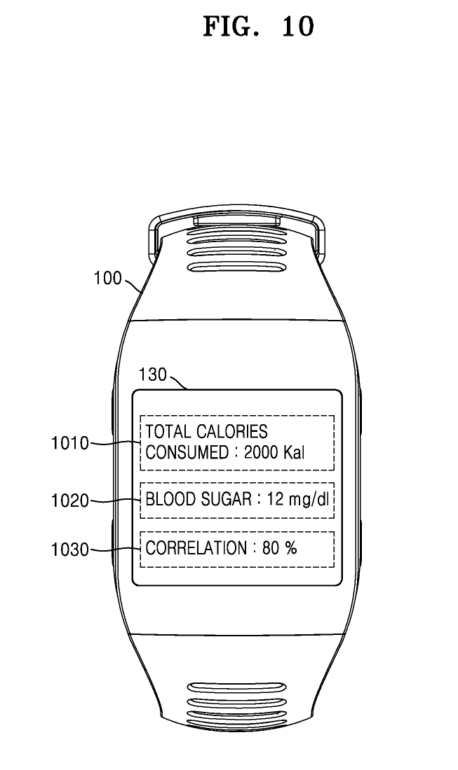

FIGS. 10 through 12 are reference diagrams for showing a correlation between a bio-signal and other information according to an exemplary embodiment. As shown in FIG. 10, the display module 130 may display total calories 1010 acquired by the subject as food information, blood sugar 1020 as bio-signal, and a correlation 1030 between the total calories 1010 and the blood sugar 1020 as a value. Then, the user may check a correlation between the total calories and blood sugar. Thus, the user may determine that blood sugar should be checked if the number of acquired calories is high.

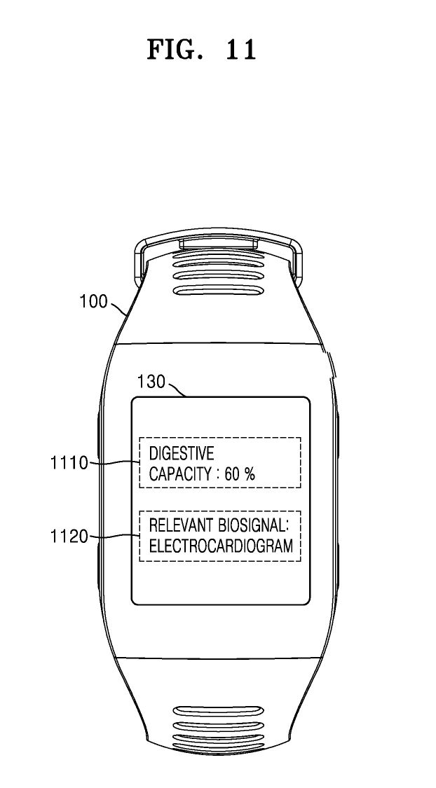

If a plurality of sensor are all operated and thus detect a subject's food information or bio-signal, overload may occur during signal processing. The user may activate just one from among the plurality of sensors. For example, the user may activate a sensor for detecting food information. The apparatus 100 for processing food information may detect food information, calculate a digestive capacity of the user, and display a result 1110 of the calculating on the display module 130 as shown in FIG. 11. The apparatus 100 for processing food information may also provide a type of the bio-signal 1120 to inform the subject that the digestive capacity has to be checked or a type of bio-signal that may be abnormal. In FIG. 11, ECG is displayed as a type of the bio-signal 1120 to inform the subject to check the digestive capacity. This is because a correlation between a digestive capacity and ECG is stored in the food information processing apparatus 100 according to an exemplary embodiment. Thus, the subject may determine that the current digestive capacity indicates an ECG problem. Thus, the user may measure the ECG by activating a sensor for detecting the ECG. The activating of the sensor for detecting bio-signal may be performed by a command received from the subject, but may also be performed automatically by the apparatus 100 for processing food information by using the correlation described above.

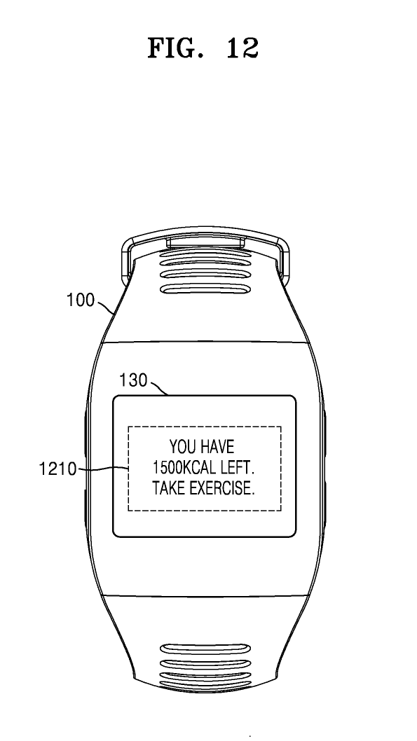

Alternately, as shown in FIG. 12, the apparatus 100 for processing food information may display information 1210 about calories that are to be consumed by the subject, or provide a method for consuming calories based on a food information pattern.

As described above, according to the one or more of the above embodiments, the method and apparatus for processing food information may improve user's convenience when compared to the conventional ones as the food information is detected by using a non-invasive method. According to the one or more of the above embodiments, the method and apparatus for processing food information may provide food information, bio-signal relating to food, or the like to a user.

In addition, other embodiments can be implemented as computer-readable code/instructions in/on a medium, e.g., a computer-readable medium, to control at least one processing element to implement any of the above-described embodiments. The medium can correspond to any medium/media permitting the storage and/or transmission of the computer-readable code.

The computer-readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as Internet transmission media. Thus, the medium may be such a defined and measurable structure including or carrying a signal or information, such as a device carrying a bitstream according to one or more embodiments of the inventive concept. The media may also be a distributed network, so that the computer-readable code may be stored/transferred and executed in a distributed fashion. Furthermore, the processing element could include a processor or a computer processor, and processing elements may be distributed and/or included in a single device.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A method of processing food information, comprising:
detecting, by a sensor, food information of food consumed by a subject from blood of the subject in a non-invasive manner; and
determining, by a processor, a digestive capacity of the subject based on the detected food information.

2. The method of claim 1, wherein the food information comprises at least one of a type of nutrients and amounts of nutrients in the food consumed by the subject.

3. The method of claim 1, wherein the determining the digestive capacity comprises:
calculating a food information pattern that shows a change in the detected food information according to a lapse of time; and
calculating the digestive capacity based on the food information pattern.

4. The method of claim 1, wherein the detecting is performed by using at least one of a Raman spectroscopy, an infrared spectroscopy, or a radio-frequency (RF) analysis.

5. The method of claim 1, further comprising displaying at least one of the food information and the digestive capacity.

6. The method of claim 1, further comprising:
detecting a bio-signal from the subject; and
calculating a correlation between the food information and the bio-signal.

7. The method of claim 6, wherein the bio-signal is detected in a non-invasive manner.

8. The method of claim 6, wherein the bio-signal comprises at least one of information about blood sugar, cholesterol, or an amount of body fat of the subject, and information about a blood pressure, electrocardiogram (ECG), ballistocardiogram (BCG), photoplethysmograph (PPG), or electromyogram.

9. The method of claim 6, wherein the correlation represents a degree of a change in the bio-signal according to a change in the food information.

10. The method of claim 6, wherein the correlation comprises a range of the food information which corresponds to a reference range of the bio-signal.

11. The method of claim 6, wherein the calculating the correlation comprises:
calculating a food information pattern according to a lapse of time by using the food information;
calculating a bio-signal pattern by using the bio-signal; and
calculating a value of the correlation between the bio-signal pattern and the food information pattern.

12. The method of claim 1, further comprising:
detecting at least one of environment information about an external environment of the subject and state information of the subject; and
calculating a correlation between the food information, and at least one of the environment information and the state information.

13. The method of claim 12, wherein the calculating the correlation comprises calculating the correlation between the food information and at least one of a temperature, a humidity, a skin moisture content rate of the subject, and a motion of the subject.

14. An apparatus for processing food information, comprising:
a first sensor configured to detect food information of food consumed by a subject from blood of the subject in a non-invasive manner; and
a processor configured to determine a digestive capacity of the subject based on the detected food information.

15. The apparatus of claim 14, wherein the food information comprises at least one of a type of nutrients and amounts of nutrients in the food consumed by the subject.

16. The apparatus of claim 14, wherein the processor is further configured to calculate a food information pattern that shows a change in the detected food information according to a lapse of time, and calculate the digestive capacity based on the food information pattern.

17. The apparatus of claim 14, wherein the first sensor is further configured to detect food information by using at least one of a Raman spectroscopy, an infrared spectroscopy, or a radio-frequency (RF) analysis.

18. The processing apparatus of claim 14, further comprising a display configured to display at least one of the food information and the digestive capacity.

19. The apparatus of claim 14, further comprising a second sensor configured to detect at least one of a bio-signal of the subject, environment information about an external environment of the subject, and state information of the subject.

20. The apparatus of claim 19, wherein the processor is further configured to calculate a correlation between a result obtained from the second sensor and the food information.
